(19) United States
(12) Patent Application Publication
(10) Pub. No.: US 2025/0118446 A1
(43) Pub. Date: Apr. 10, 2025

(54) SYSTEM AND METHOD FOR PREDICTING HEPATOCELLULAR CARCINOMA RECURRENCE RISK OF A PATIENT AFTER A SURGERY

(71) Applicants: The University of Hong Kong, Hong Kong (HK); The Education University of Hong Kong, Hong Kong (HK)

(72) Inventors: Jianliang LU, Hong Kong (HK); Wai Kay Walter SETO, Hong Kong (HK); Man Fung Richard YUEN, Hong Kong (HK); Keith Wan Hang CHIU, Hong Kong (HK); Wan Hin Rex HUI, Hong Kong (HK); Homing CHENG, Hong Kong (HK); Philip Leung Ho YU, Hong Kong (HK); Chenglu WANG, Hong Kong (HK)

(21) Appl. No.: 18/907,615

(22) Filed: Oct. 7, 2024

Related U.S. Application Data
(60) Provisional application No. 63/588,757, filed on Oct. 9, 2023.

(51) Int. Cl. G16H 50/30; G06T 7/00

(52) U.S. Cl. CPC G16H 50/30; G06T 7/0014; G06T 2207/10081; G06T 2207/20081; G06T 2207/30056

(57) ABSTRACT

The present invention relates to methods for predicting the recurrence risk of hepatocellular carcinoma (HCC). Specifically, it proposes a deep learning model capable of integrating information from different phases of CT images and clinical data to predict the risk of HCC recurrence within 1 to 5 years after treatment. Experimental results demonstrate that these models outperform traditional prediction methods based on histological microvascular invasion (MVI) in predicting HCC recurrence risk.

### CROSS-REFERENCE TO RELATED APPLICATIONS

The present application claims the priority from the U.S. provisional patent application Ser. No. 63/588,757 filed Oct. 9, 2023, and the disclosure of which is incorporated herein by reference in its entirety.

### FIELD OF THE INVENTION

The present invention pertains to the field of medical technology, specifically in the area of the treatment and prediction of liver cancer, with a focus on hepatocellular carcinoma (HCC), which is the predominant type of primary liver cancer.

### BACKGROUND OF THE INVENTION

Primary liver cancer is the seventh most common cancer and the third most common cause of cancer mortality worldwide, with HCC being the predominant type of primary liver cancer. Therapeutic strategies including liver resection, liver transplantation and local ablation are feasible first-line treatment for early-stage HCC, yet liver resection remains as the main curative treatment in the Asia-Pacific region, primarily due to the scarcity of organ donation. Despite the experience in curative surgery, early HCC recurrence within 2 years is common, and the overall recurrence risk within 5-years is over 50%. The ability to predict post-operative recurrence is paramount for risk stratification and may affect surveillance strategies. High-risk patients may also be candidates for neoadjuvant or adjuvant treatment, although most of such strategies remain in clinical trials.

Medical data including clinical records, computed tomography (CT) images, magnetic resonance imaging (MRI) and multispectral imaging (MSI) have been used in the modeling of predict the risk of HCC recurrence. For instance, Shiyin Hong et al. reported an artificial intelligence model based on 6 tumors and related region for prediction of HCC recurrence risk¹. Ailian Liu et al.² and Sijie Niu et al.³ reported a machine learning and a deep-learning model on MRI image, respectively. Huang, Y. et al.⁴ and Zeng, J. et al.⁵ aimed to identify patient risk after surgical resection by machine learning and Cox model on clinical variables respectively.

For those models aimed to a higher accuracy by combine CT image and clinical variables, they usually extracted features from region of interest of CT image and built a machine learning model or Cox model for the prediction of survival risk^{6,7,8}.

Histological features are usually involved in the training dataset, making the model post-operative predictable⁹. The limitation of extracting features from region of interests is that limited information can be extracted, and potential tumor or risk region may be missed by human error in the labeling of the regions.

Therefore, there is a need in the art for more advanced and accurate predictive models that can minimize human error by analyzing entire imaging datasets rather than limited regions of interest.

### SUMMARY OF THE INVENTION

The present invention is aimed to calculate recurrent risk of HCC after curative surgery. As the deep-learning proved its capability in HCC studies, the present invention develops a novel deep-learning model on pre-operative CT images for predicting HCC recurrence after curative surgery.

The present invention facilitates prognosis during the pre-operative phase, and forecast both early and late recurrence. To improve the effectiveness of extracting feature from whole CT images, two strategies are adopted: apply liver segmentation to identify the slices have contents of liver; and applying a two branches neural network architecture with hepatic arterial phase and porto-venous phase as input, respectively, to increase the contrast for better identifying of tumor lesions.

In addition, the present invention also presents several adapted editions of Recurr-NET, facilitating prognostication under different scenarios: using CT data exclusively, combining CT with essential clinical data, and integrating CT with comprehensive clinical data.

In a first aspect, the present invention provides a method for predicting HCC recurrence risk of a patient after a surgery. The method includes obtaining one or more pre-operative CT images from the patient's liver, wherein the CT images include at least hepatic arterial phase and porto-venous phase images including features associated with tumor lesions; feeding the one or more pre-operative CT images into a multiphasic deep-learning model and training the multiphasic deep-learning model with the one or more pre-operative CT images; generating a joint image-based representation of the pre-operative CT images by concatenating corresponding extracted image features; and calculating a predicted risk score of the HCC based on the extracted image features for planning post-operative treatment and monitoring within a predetermined time period after the surgery.

In another embodiment, the method further includes the step of segmenting the liver region in the one or more pre-operative CT images using a liver segmentation model before feeding the one or more pre-operative CT images into the multiphasic deep-learning model.

In one embodiment, the multiphasic deep-learning model is configured to extract image features from the one or more pre-operative CT images.

In one embodiment, the multiphasic deep-learning model includes a first residual network architecture applied to the hepatic arterial phase images, a second residual network architecture applied to the porto-venous phase images, and a multiphasic residual-network random survival forest (RSF) model. The RSF model combines the extracted image features to generate the predicted risk score. The RSF model uses a log-rank splitting rule and a bootstrap method for analyzing right-censored survival data.

In one embodiment, each of the two residual network architectures includes at least one stem block, at least one convolution block, and at least one identification (ID) block.

In another embodiment, the method further includes the step of inputting pre-operative clinical parameters of the patient into the multiphasic deep-learning model.

In one embodiment, the RSF model combines the extracted image features with the pre-operative clinical parameters to generate the predicted risk score.

In one embodiment, the pre-operative clinical parameters are selected from age, sex, hepatitis B surface antigen status, hepatitis C virus antibody status, history of fatty liver, alpha fetoprotein level, and Model for End-Stage Liver Disease (MELD) score.

In another embodiment, the pre-operative clinical parameters further include smoking status, comorbidities, use of antiviral therapy for hepatitis B, baseline blood test results including platelet count, prothrombin time, albumin, alpha fetoprotein levels, or a combination thereof.

In one embodiment, the multiphasic deep-learning model is optimized using the Adam optimizer with batch normalization.

In one embodiment, the predetermined time period ranges from 1 to 5 years.

In a second aspect, the present invention provides a system for predicting a risk of hepatocellular carcinoma recurrence. The system includes a processor, a memory storing instructions, and a user interface to display an output of the predicted risk score. The memory storing instructions, when executed by the processor, cause the system to perform the following steps: obtaining one or more pre-operative computed tomography (CT) images from the patient's liver, wherein the CT images include at least hepatic arterial phase and porto-venous phase images that feature characteristics associated with tumor lesions; feeding the one or more pre-operative CT images into a multiphasic deep-learning model and training the multiphasic deep-learning model with the one or more pre-operative CT images; generating a joint image-based representation of the pre-operative CT images by concatenating corresponding image features; and calculating a predicted risk score of the HCC based on a visual information of the joint image-based representation for planning post-operative treatment and monitoring within a predetermined time period after the surgery.

In one embodiment, the memory storing instructions further cause the system to segment the liver region in the one or more pre-operative CT images using a liver segmentation model before feeding the one or more pre-operative CT images into the multiphasic deep-learning model.

In one embodiment, the multiphasic deep-learning model includes an image model based on two residual network architectures and a multiphasic residual-network RSF model that combines the extracted image features to generate the predicted risk score. The two residual network architectures are each applied separately to the hepatic arterial phase and porto-venous phase images.

In one embodiment, each of the two residual network architectures includes at least one stem block, at least one convolution block, and at least one identification (ID) block.

In one embodiment, the RSF model uses a log-rank splitting rule and a bootstrap method for analyzing right-censored survival data.

In one embodiment, the memory storing instructions further cause the system to inputting pre-operative clinical parameters of the patient into the multiphasic deep-learning model.

In one embodiment, the pre-operative clinical parameters include age, sex, hepatitis B surface antigen status, hepatitis C virus antibody status, history of fatty liver, alpha fetoprotein level, MELD score, or a combination thereof.

In another embodiment, the pre-operative clinical parameters further include smoking status, comorbidities, use of antiviral therapy for hepatitis B, baseline blood test results including platelet count, prothrombin time, albumin, alpha fetoprotein levels, or a combination thereof.

The present invention has the following advantages:
(1) Both CT scans and all clinical parameters used in the model training are pre-operative, excluding any post-operative information, thus enabling prognostication in the pre-operative stage;
(2) The model takes full CT images as input, rather than extracting features from labeled tumor regions, eliminating the potential for valuable information to be missed and reducing the risk of human errors;
(3) The model's robustness in predicting HCC recurrence has been tested on an internal validation dataset consisting of 135 samples from Hong Kong and an external testing dataset comprising 560 samples from Taiwan.

### BRIEF DESCRIPTION OF THE DRAWINGS

Embodiments of the invention are described in more details hereinafter with reference to the drawings, in which:

FIG. 1 shows a schematic diagram of the system of the present invention;

FIG. 2 shows an architecture of the Recurr-NET^{CT}. Image embedding block represents the whole structure of the network, constructed by the stem block, the conv block, and the ID block;

FIG. 3 shows an architecture of the RSF model;

FIGS. 4A-4D show cumulative probability of recurrence for both internal validation and external testing cohorts at year 2 and year 5; and

FIGS. 5A-5D show the model's ability to predict all-cause mortality in patients with HCC for both internal validation and external testing cohorts at year 2 and year 5.

### DETAILED DESCRIPTION

In the following description, system and methods for predicting HCC recurrence risk of a patient after a surgery are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

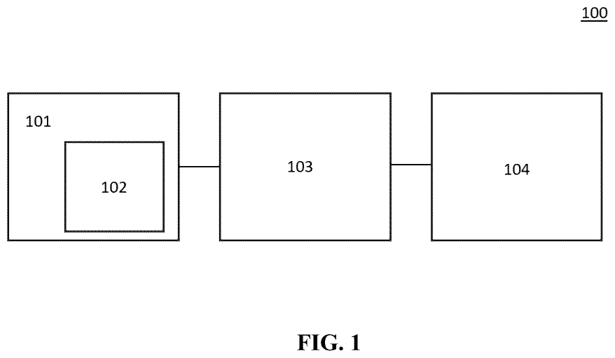

As shown in FIG. 1, the system 100 includes a processor 101 and a memory storing instructions 102. When executed by the processor 101, the instructions 102 cause the system 100 to perform a series of steps aimed at predicting HCC recurrence. The core functionality revolves around processing pre-operative CT images and combining the extracted image features with relevant clinical data to generate an accurate prediction of recurrence risk. Finally, the system 100 includes a user interface 104 to display the output of the predicted risk score, providing clinicians with easy access to critical information that can influence treatment decisions.

The system 100 begins by obtaining pre-operative CT images from the patient's liver. These CT images include at least the hepatic arterial phase and portal venous phase, both of which are critical in identifying characteristics associated with tumor lesions. The CT scans typically cover the region from the lung base to the iliac crest, ensuring that all relevant liver tissues are included.

The memory storing instructions 102 further cause the system 100 to perform liver segmentation on the pre-operative CT images before they are processed by a multiphasic deep-learning model 103. This segmentation isolates liver-specific slices from the full CT scan using a segmentation model trained on public datasets, such as the Liver Tumor Segmentation Challenge (LiTS). By focusing on liver regions, the system 100 enhances the accuracy of the deep-learning model 103, ensuring that only relevant areas are analysed for predicting recurrence.

The system 100 includes a multiphasic deep-learning model 103 that processes the segmented CT images. This model comprises an image model based on two residual network architectures, with each network architecture applied separately to the hepatic arterial phase and portal venous phase images. These two network branches extract corresponding image features from the different phases, which are then concatenated to generate a joint image-based representation.

The RSF model combines the extracted image features to generate a predicted risk score using a log-rank splitting rule and a bootstrap method to analyze right-censored survival data, ensuring that the predicted risk score accurately reflects the patient's recurrence risk over a specified time period. The model is trained on a dataset that includes both internal validation and external testing cohorts. The loss function used is based on the Cox proportional hazard function with regularization to account for recurrence data.

The memory storing instructions 102 also cause the system 100 to input pre-operative clinical parameters of the patient into the multiphasic deep-learning model 103. These clinical parameters are combined with the image-based features to improve the overall predictive accuracy of the system 100.

The clinical data may include essential factors such as the patient's age, sex, hepatitis B surface antigen status, hepatitis C virus antibody status, history of fatty liver, alpha fetoprotein levels, and MELD score. In some cases, additional clinical data such as smoking status, comorbidities, and baseline blood test results (including platelet count, prothrombin time, albumin levels, and alpha fetoprotein levels) are also incorporated.

After processing the CT images and clinical data, the system 100 calculates a predicted risk score for HCC recurrence. This risk score is used to guide post-operative treatment planning and ongoing monitoring of the patient. Finally, the system 100 outputs the predicted risk score to the user interface to display 104, providing clinicians with easy access to critical information that can influence treatment decisions. This system 100 provides a powerful tool for pre-operative prognostication of HCC outcomes, enabling more informed clinical decisions and potentially improving patient outcomes through targeted post-operative interventions.

Moreover, the method of this invention describes how to use a deep learning model to integrate information from different phases of CT images, extract features related to tumors, and ultimately predict the recurrence risk of HCC.

The method of the present invention involves training three distinct versions of a deep learning model to predict the recurrence risk of HCC from years 1 to 5 post-treatment. These models include: Recurr-NET^{CT}, Recurr-NET^{LITE}, and the full Recurr-NET model, which will be detailed below.

### I. Recurr-NET^{CT}

Recurr-NET^{CT} is a deep-learning model that utilizes only pre-operative CT images for predicting HCC recurrence.

The CT images are stored in Digital Imaging and Communications in Medicine (DICOM) format, which is a standard for handling, storing, and transmitting medical imaging information. The CT images are segmented to focus on liver-specific slices using a liver segmentation model trained on public datasets such as the LiTS (Liver Tumor Segmentation Challenge). These CT images are then normalized and resized to a standard dimension of 512×512×32. Data augmentation techniques, including 15-degree rotations, are applied to enhance the model's robustness. The deep-learning model is constructed based on a residual network (ResNet) architecture, utilizing the arterial phase and portal venous phase of the CT images. The model generates a joint image-based representation by extracting and concatenating features from the two phases. The training is optimized using the Adam optimizer with batch normalization for faster convergence.

AUROC (Area Under the Receiver Operating Characteristic Curve) is a metric used to evaluate the performance of a binary classification model. It measures the ability of the model to distinguish between classes at various threshold settings. The AUROC value ranges from 0 to 1, where a value of 0.5 indicates no discriminative ability, meaning the model is no better than random guessing, and a value of 1.0 indicates perfect classification, meaning the model can perfectly distinguish between the classes. The AUROC values reflect the model's performance in predicting HCC recurrence, with higher AUROC values indicating better performance in identifying recurrence.

Recurr-NET^{CT} demonstrated reliable performance in predicting HCC recurrence within 1 to 5 years post-surgery. In the internal validation cohort, the Recurr-NET^{CT} achieve a performance from 1-5 years with AUROC of 0.756 to 0.796, while in the external cohort, it achieves AUROC values ranging from 0.693 to 0.755. The high and stable AUROCs achieve from a relatively large external testing cohort illustrated the robustness of the present model.

### II. Recurr-NET^{LITE}

Recurr-NET^{LITE} builds upon Recurr-NET^{CT} by incorporating additional basic clinical parameters alongside the pre-operative CT images.

The basic clinical parameters may include age, sex, hepatitis B surface antigen, hepatitis C virus antibody, history of fatty liver on imaging, alpha fetoprotein, and model for MELD score. All the clinical parameters are pre-operative data.

Similar to Recurr-NET^{CT}, the CT images are segmented, normalized, and resized, and then fed into a ResNet-based architecture. The extracted image features are combined with the clinical parameters to enhance the model's prognostic capabilities.

The Recurr-NET^{LITE} achieves slightly improved predictive performance compared to Recurr-NET^{CT}. In the internal validation cohort, it achieves a diagnostic accuracy in predicting HCC recurrence from years 1-5 with AUROC of 0.742 to 0.811, while in the external cohort, the AUROC values range from 0.711 to 0.760. The integration of clinical data with image features enhances the model's accuracy in predicting recurrence, providing more nuanced risk stratification for patients.

### III. Full Recurr-NET

The full Recurr-NET model is an advanced version that incorporates both pre-operative CT images and comprehensive clinical parameters.

The full Recurr-NET is a multiphasic residual-network RSF deep-learning model. In addition to the basic clinical parameters used in Recurr-NET^{LITE}, the full Recurr-NET also includes factors such as smoking status, comorbidities, use of antiviral therapy for hepatitis B, and additional blood test results (e.g., platelet count, prothrombin time, albumin levels).

The full Recurr-NET is trained based on multiphasic contrast CT liver images from Chinese patients with resected histology-confirmed HCC from four centers in Hong Kong (Internal cohort). The internal cohort is randomly divided into 80% for training and 20% for internal validation. External validation of Recurr-NET in an independent cohort from Taiwan is also performed. The area-under-curve (AUC), positive-and negative-predictive values (PPV/NPV) of Recurr-NET is compared against microvascular invasion (MVI) through diagnostic accuracy and survival analysis respectively.

For image and data processing, the CT images are obtained from major global CT manufacturers (Siemen, GE, Philips and Toshiba) and stored in Digital Imaging and Communications in Medicine (DICOM) format. The Hounsfield units (HU) of the images are windowed to a range of [−160, 240], and the images are normalized to a range of [−0.5, 0.5] to remove extraneous features. The raw CT image size is 512 (height)×512 (width)×N, where N denotes the number of slices. The CT scans span from the lung base to the iliac crest and include three phases (non-contrast phase, hepatic arterial phase and porto-venous phase). To enhance the performance of deep learning models, liver-related CT slices are extracted using a liver segmentation model trained on the public dataset LiTS (Liver Tumor Segmentation Challenge).

To facilitate computation, the image size of CT scans is resized to 512×512×32. Two data augmentation methods are employed: 15-degree clockwise rotation and 15-degree anti-clockwise rotation. Multiple imputation handles missing clinical data. Continuous variables are normalized to the [0,1], and discrete variables are encoded as one-hot vectors.

Deep learning is performed on PyTorch 1.12.1, facilitated by a computational platform powered by NVIDIA Tesla V100 graphic processing units and Intel(R) Xeon(R) Gold 6146 CPU@3.20 GHz with 12, 256 GB DDR4 Memory. Using the processed triphasic CT scans and pre-operative clinical data, Recurr-NET, a multimodal multiphasic residual-network RSF deep-learning model, is developed to predict the risk of HCC recurrence within 5 years for each patient.

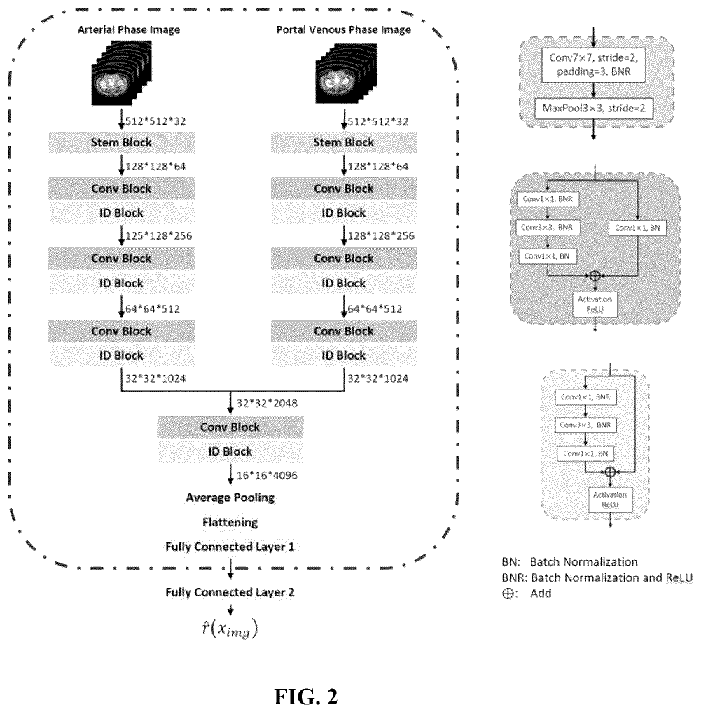

For model architecture, the Recurr-NET consists of an image model based on the residual network (ResNet) structure, and a RSF model. FIG. 2 shows the architecture of the Recurr-NET^{CT}. First, patients' arterial phase and portal venous phase images are fed into two branches of the ResNet architectures to obtain corresponding image features. The separate encoding path of each branch is composed of one stem block and three residual blocks, which including a convolution block and an ID block.

Each branch begins with a stem block, which typically includes an initial convolutional layer and batch normalization. This block processes the raw image data and prepares it for deeper layers.

After the stem block, the image passes through a series of convolutional blocks, which are responsible for extracting increasingly abstract features from the image. These blocks consist of convolutional layers followed by activation functions (e.g., ReLU) and batch normalization. The convolution blocks play a critical role in learning spatial hierarchies in the image data.

Each convolution block is followed by an ID block, which includes skip connections (residual connections) that help mitigate the vanishing gradient problem during training. These skip connections allow the network to learn more effectively by preserving gradients across layers.

Once the images from both phases have passed through their respective residual networks, the extracted features are concatenated. This joint image-based representation integrates information from both the hepatic arterial and portal venous phases, enhancing the model's ability to detect subtle patterns associated with tumor lesions and other risk factors for recurrence. A risk score \hat{r}(x_{img}) is generated based on visual information transmitted through the Fully Connected block. In the initial stage, Adam with an initial learning rate (lr) of 0.001 is adopted to optimize the training process. Batch normalization is used after each convolution layer, which leads to faster convergence via ensuring that the activation outputs of convolutional layers are in proper.

Meanwhile, batch normalization makes it possible each layer to learn independently of the others and reduces data lost between processing layers. The average negative log partial likelihood of the Cox proportional hazard function with regularization is used as the loss function:

$$l ( β ) : = - \frac{1}{{N}_{E = 1}} {\sum }_{i : {E}_{i} = 1} ( {\hat{h}}_{β} ( {x}_{i} ) - log {\sum }_{j ∈ ℛ ( {T}_{i} )} {e}^{{\hat{h}}_{β} ( {x}_{j} )} ) + λ \cdot {‖ β ‖}_{2}^{2} ,$$

where N_{E=1} is the number of patients with recurrence records. The values Tᵢ, Eᵢ and xᵢ denotes the event time, event indicator, and baseline data for the i^{th} observation respectively. The risk set [symbol] (t)={i:Tᵢ≥t} represents the set of patients still at risk of recurrence at time t. ĥ_{β}(x) is the log-risk parameterized by β.

Following the principles of transfer learning, the 64-dimensional vector obtained from Fully Connected Layer 1 of the image model (FIG. 2) is employed as the representation of image information.

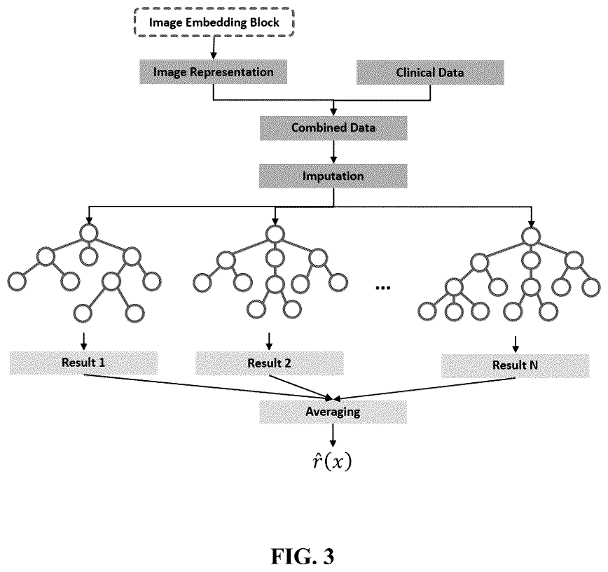

As needed, this representation is combined with patients' clinical data before being fed into the RSF model (FIG. 3). The RSF is a tree model for analyzing right-censored survival data. It uses the log-rank splitting rule, which splits nodes by maximizing differences between nodes. The bootstrap method is used in the model to randomly select samples from the original data with replacement, establishing a sample subset and out-of-bag (OOB) dataset. Secondly, it randomly selects features for each sample to construct its corresponding survival tree. The Nelson-Aalen method is used to estimate the total cumulative risk of the RSF model. The OOB data is used to estimate prediction accuracy. The RSF model finally predict each patient's risk score \hat{r}(x), and the risk scores is fitted into a Kaplan-Meier estimator to get the corresponding survival curve.

Kaplan-Meier estimator which based on empirical and censoring data is used to calculate the survival function of each patient. The probability that the patient will not be recurrent at time t is defined as:

$$\hat{S} ( t ) = {π}_{{t}_{i} < t} \frac{{n}_{i} - {d}_{i}}{{n}_{i}} ,$$

where dᵢ is the number of recurrence cases at the time tᵢ, and nᵢ is the number of patients at risk of recurrence just before the time tᵢ.

The survival possibility S(tⱼ) at time tⱼ can be calculated iteratively as follows:

$$\{ \begin{matrix}S ( {t}_{0} ) = 1 \\ S ( {t}_{j + 1} ) = S ( {t}_{j} ) \cdot ( 1 - \frac{{d}_{j}}{{n}_{j}} )\end{matrix} ,$$

where Youden index is used to choose the cut-off point and decide recurrent or not.

In the internal validation cohort, the full Recurr-NET achieves excellent diagnostic accuracy in predicting HCC recurrence from years 1-5 with AUROC of 0.770 to 0.857, while in the external cohort. it achieves AUROC values ranging from 0.758 to 0.798.

The combination of comprehensive clinical data with the CT image features allows the full Recurr-NET model to outperform both Recurr-NET^{CT} and Recurr-NET^{LITE}, providing a highly accurate tool for pre-operative prognostication.

### EXAMPLE

Internal Validation and External Testing Conducted on Full Recurr-NET Compare with MVI Predicted Recurrence

Recurrence can occur in over 70% of HCC patients within 5 years after curative resection. While histological MVI predicts recurrence, it is ascertained from resected specimens and cannot provide pre-operative prognostication.

The present invention provides a deep learning model using pre-operative CT and clinical parameters for predicting HCC recurrence. Chinese patients with resected histology-confirmed HCC are recruited from four centers in Hong Kong and Taiwan are used for training, internal validation, and external testing, respectively.

This analysis included 1,231 patients (83.1% male, age 62.4+/−10.7 years, median follow-up 65.1 months). 536 (43.9%), 135 (11.2%) and 560 (44.9%) patients are in the training, internal validation, and external testing cohorts respectively. The cumulative HCC recurrence rate at years 2 and 5 are 41.8% and 56.4% respectively.

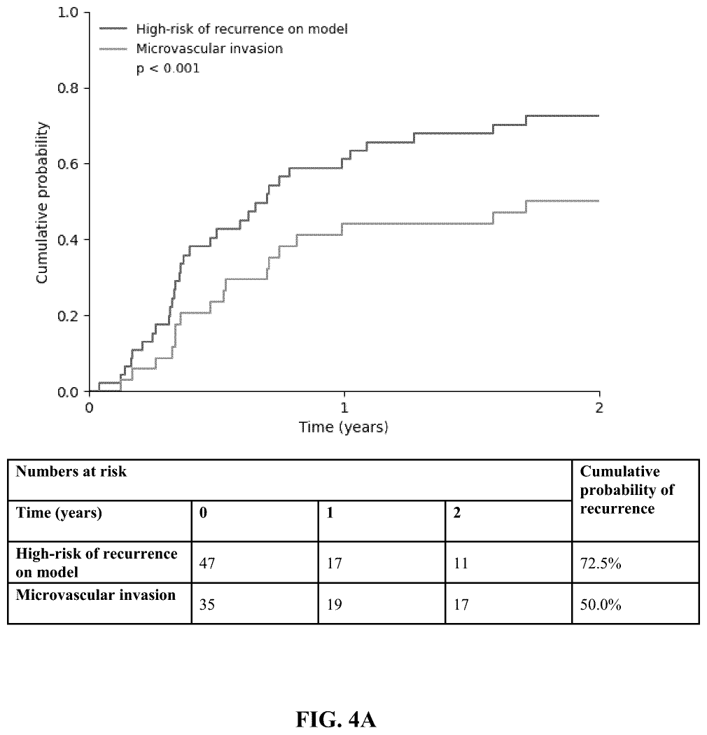

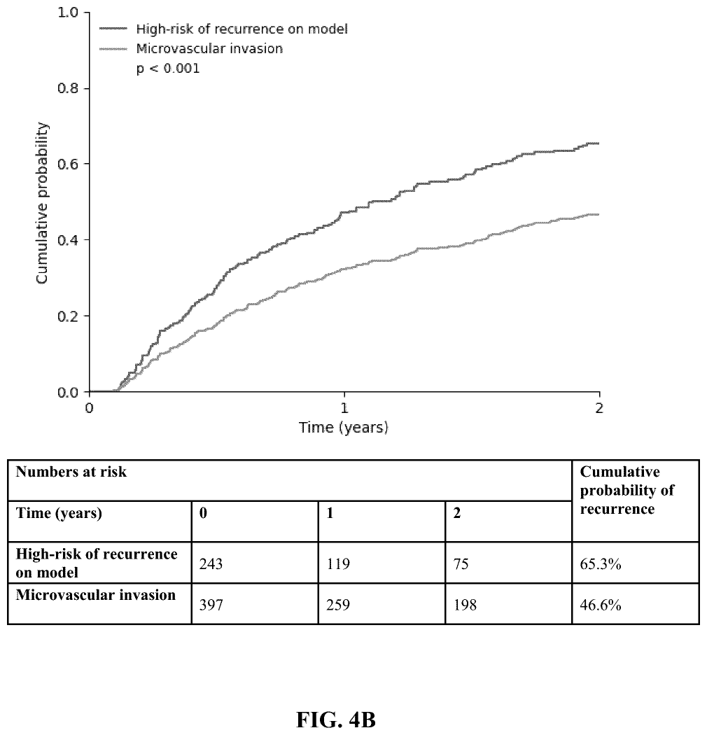

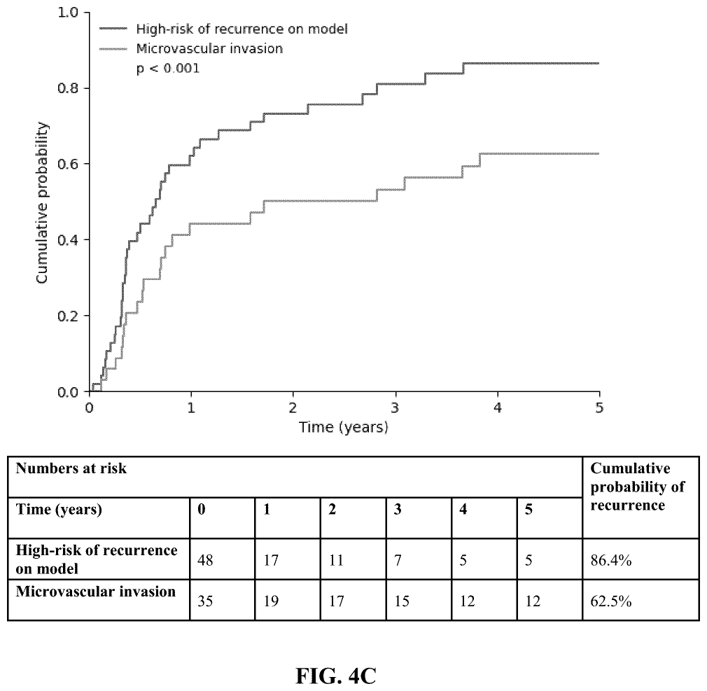

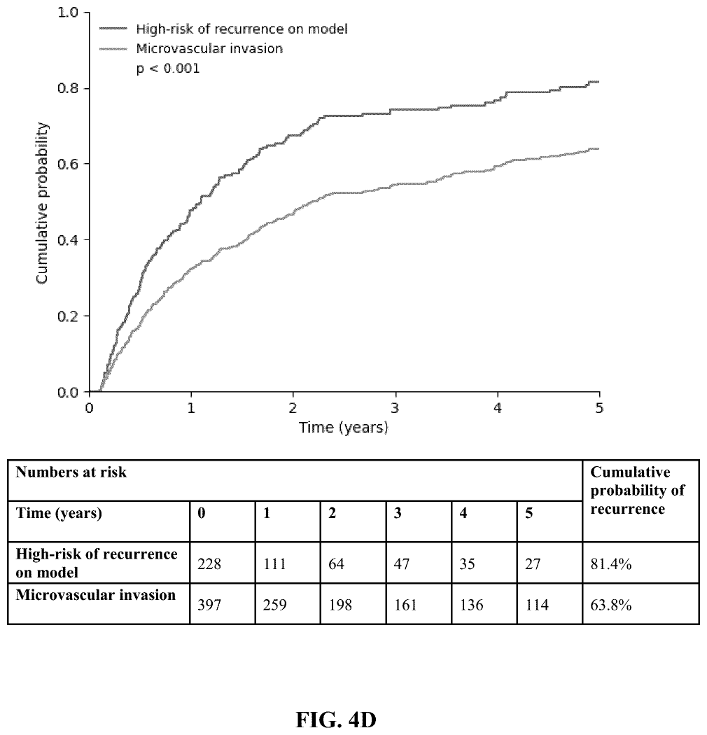

FIGS. 4A-4D illustrate the cumulative probability of hepatocellular carcinoma (HCC) recurrence for both internal validation and external testing cohorts at year 2 and year 5. These figures compare the performance of the full Recurr-NET model with traditional histological MVI in predicting HCC recurrence. FIG. 4A shows the cumulative probability of HCC recurrence at year 2 for the internal validation cohort. This graph highlights Recurr-NET's ability to predict early recurrence, demonstrating superior performance compared to MVI in identifying high-risk patients within two years post-surgery. FIG. 4B presents the cumulative probability of HCC recurrence at year 2 for the external testing cohort. Similar to FIG. 4A, it showcases Recurr-NET's enhanced predictive accuracy over MVI, but in an independent patient population, confirming the model's robustness. FIG. 4C illustrates the cumulative probability of HCC recurrence at year 5 for the internal validation cohort. This long-term prediction shows that full Recurr-NET continues to outperform MVI in stratifying recurrence risk, indicating the model's effectiveness in forecasting late recurrence. FIG. 4D displays the cumulative probability of HCC recurrence at year 5 for the external testing cohort. Like FIG. 4C, this figure demonstrates that full Recurr-NET maintains superior predictive performance over MVI in a different cohort over a five-year period.

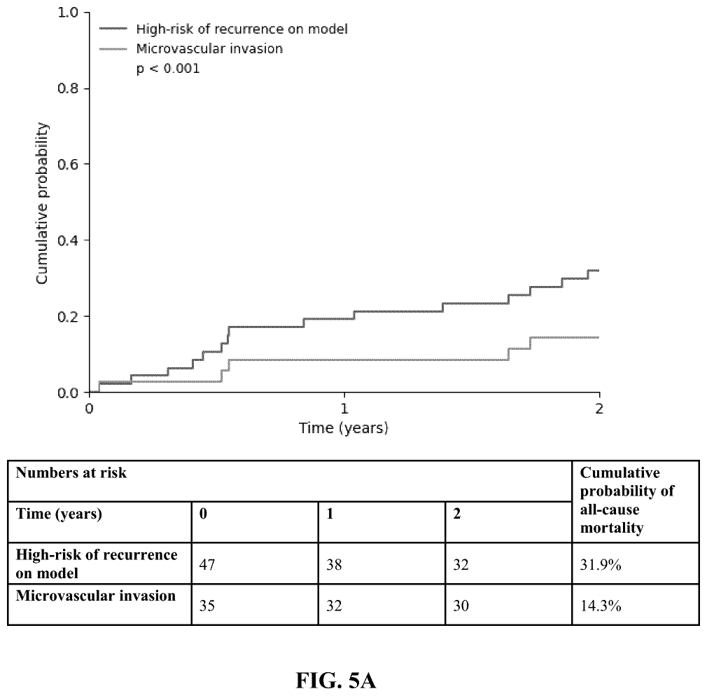

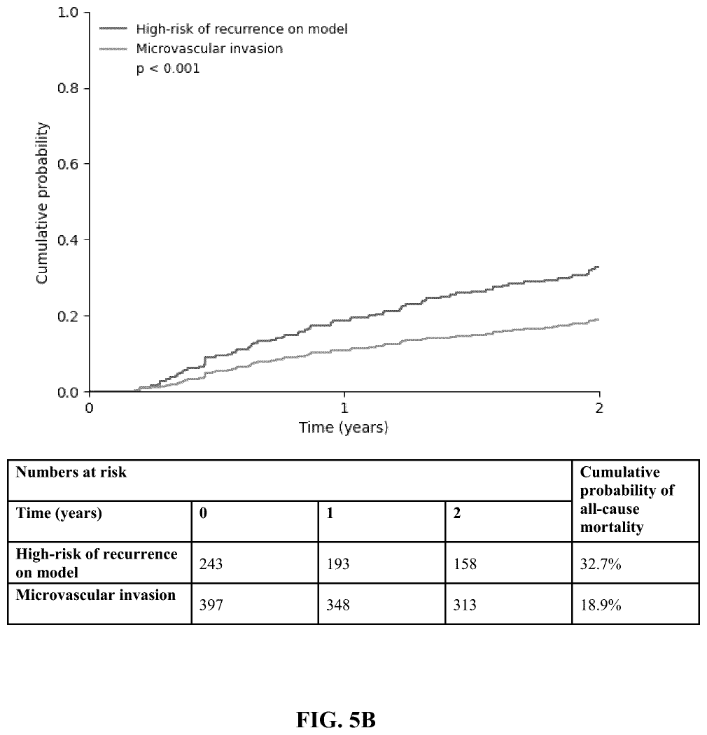

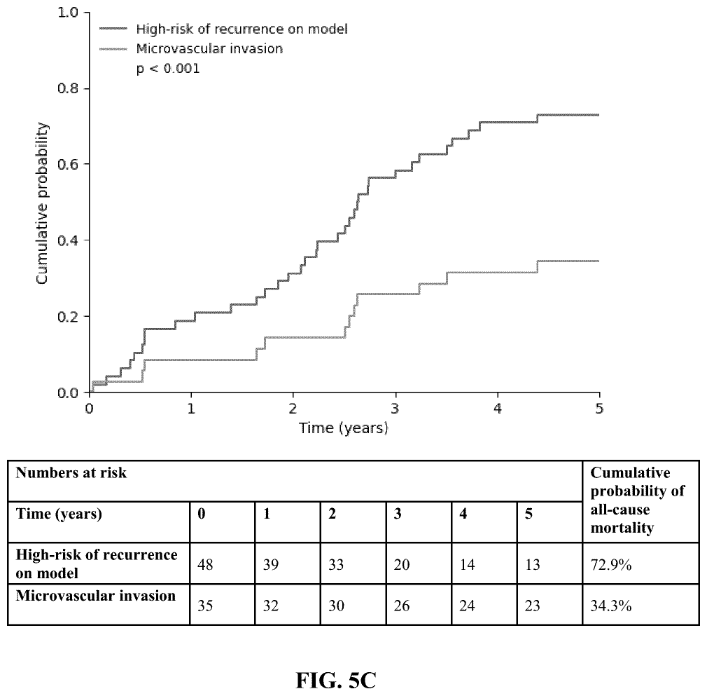

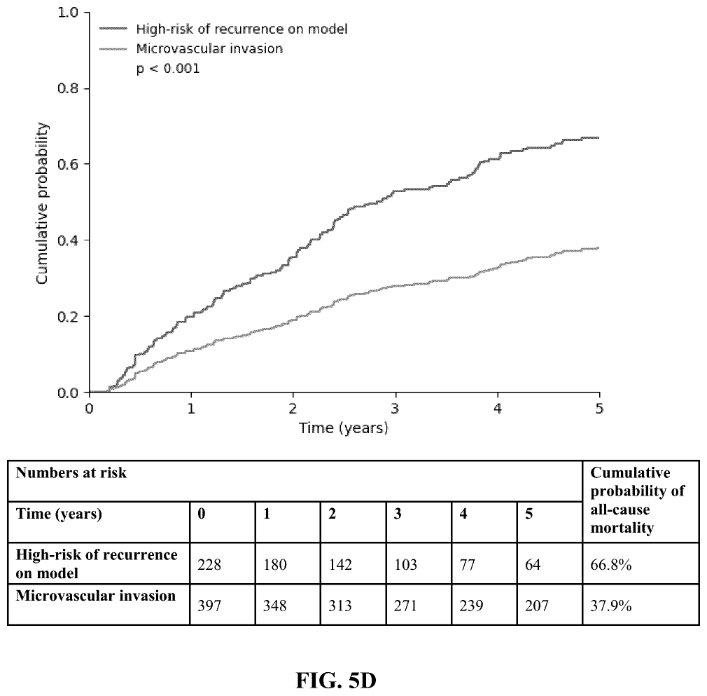

FIGS. 5A-5D provide a comparison between the predictions made by the full Recurr-NET model and those based on histological MVI, a traditional method for assessing risk. FIG. 5A shows the cumulative probability of all-cause mortality at year 2 for the internal validation cohort. The graph compares the performance of Recurr-NET with MVI, highlighting Recurr-NET's superior ability to stratify patients by their mortality risk within two years post-surgery. FIG. 5B presents the cumulative probability of all-cause mortality at year 2 for the external testing cohort. Similar to FIG. 5A, this graph demonstrates that Recurr-NET outperforms MVI in predicting mortality, but with data from a different, independent patient cohort. FIG. 5C illustrates the cumulative probability of all-cause mortality at year 5 for the internal validation cohort. This longer-term prediction shows that full Recurr-NET continues to provide more accurate stratification of mortality risk compared to MVI at the five-year mark. FIG. 5D displays the cumulative probability of all-cause mortality at year 5 for the external testing cohort. This figure reinforces the findings in FIG. 5C, showing that full Recurr-NET maintains its superior predictive performance over MVI even in an external patient population over a five-year period.

In the internal validation cohort, full Recurr-NET achieves an AUC of 0.857 (95% CI 0.783-0.921, PPV 0.744, NPV 0.807) and 0.770 (95% CI 0.681-0.849, PPV 0.884, NPV 0.543) for predicting recurrence at years 2 and 5, significantly outperforming the predictive value of MVI (year 2 AUC 0.570 [95% CI 0.491-0.654], PPV 0.500, NPV 0.670; year 5 AUC 0.518 [95% CI 0.44-0.594], PPV 0.636, NPV 0.407) (Both p<0.01).

In the external testing cohort, full Recurr-NET achieved an AUC of 0.781 (95% CI 0.735-0.819, PPV 0.668, NPV 0.789) and 0.760 (95% CI 0.718-0.801, PPV 0.863, NPV 0.513) for predicting recurrence at years 2 and 5, significantly outperforming MVI (year 2 AUC 0.605, 95% CI 0.568-0.643, PPV 0.476, NPV 0.769; year 5 AUC 0.557, 95% CI 0.515-0.597, PPV 0.678, NPV 0.459) (Both p<0.05). In both internal validation and external testing, full Recurr-NET is superior to MVI in stratifying recurrence risks at year 2 (Internal: 72.5% in full Recurr-NET vs 50.0% in MVI; External: 65.3% in full Recurr-NET vs 46.6% in MVI; both p<0.001, FIGS. 4A-4B) and year 5 (Internal: 86.4% in full Recurr-NET vs 62.5% in MVI; External: 81.4% in full Recurr-NET vs 63.8% in MVI; both p<0.001, FIGS. 4C-4D).

A comparable pattern is observable in the internal validation and external testing cohorts for all-cause mortality (data available in both internal and external cohorts), where full Recurr-NET is superior to MVI in predicting all-cause mortality at year 2 (Internal: 31.9% vs 14.3%; External: 32.7% vs 18.9% both p<0.001, FIGS. 5A-5B) and year 5 (Internal: 72.9% vs 34.3%; External: 66.8% vs 37.9%; both p<0.001; FIGS. 5C-5D).

In conclusion, full Recurr-NET is superior to MVI in predicting carly and late HCC recurrence, and has potential to emerge as a novel tool for pre-operative prognostication of HCC outcomes,

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

“Hepatocellular carcinoma (HCC)” is a type of liver cancer that originates in the hepatocytes, which are the main cells of the liver. It's the most common primary liver cancer and is often associated with chronic liver disease, including cirrhosis and hepatitis B or C infection.

The term “pre-operative” refers to the period before a surgical procedure is performed. In terms of timing, it typically means any time from the initial diagnosis of a condition up to the day of the surgery. For imaging like CT scans, “pre-operative” usually means that the images are taken within a few weeks to a few months before the surgery, depending on the nature of the condition and the urgency of the procedure.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### REFERENCES: THE DISCLOSURES OF THE FOLLOWING REFERENCES ARE INCORPORATED BY REFERENCE

1. Shiyin Hong, W. D., Mengxin Tian, Weiren Liu, Zheng Tang, Kun Qian, Zhixun Wang, Hao Zhou, Yucheng Guo, Jintao Qiu, AI-based analysis method and system for prognosis of postoperative recurrence of early hepatocellular carcinoma. 2021 Jun. 29: CN113591919A.
2. Ailian Liu, Y. G., Yin Zhao, Nan Wang, Xin Li, Jinjun Wu, Tingfan Wu, Xinhe Zhang, Imaging omics feature processing method for predicting recurrence of hepatocellular carcinoma after surgical resection based on multi-modal MRI (magnetic resonance imaging) images. 2019 Dec. 11: CN110916666A.
3. Sijie Niu, F. L., Xizhan Gao, Yiqi Wang, Qiang Liu, Jiwen Dong, MR image liver cancer postoperative recurrence risk prediction method and system based on deep learning. 2021 Apr. 28: CN113180633A.
4. Huang, Y., et al., Development and Validation of a Machine Learning Prognostic Model for Hepatocellular Carcinoma Recurrence After Surgical Resection. Front Oncol, 2020. 10: p. 593741.
5. Zeng, J., et al., Development of a machine learning model to predict early recurrence for hepatocellular carcinoma after curative resection. Hepatobiliary Surg Nutr, 2022. 11 (2): p. 176-187.
6. Zhou, Y., et al., CT-based radiomics signature: a potential biomarker for preoperative prediction of early recurrence in hepatocellular carcinoma. Abdom Radiol (NY), 2017. 42 (6): p. 1695-1704.
7. Ji, G. W., et al., Machine-learning analysis of contrast-enhanced CT radiomics predicts recurrence of hepatocellular carcinoma after resection: A multi-institutional study. EBioMedicine, 2019. 50: p. 156-165.
8. Liu, Y., et al., CT radiomics combined with clinical variables for predicting the overall survival of hepatocellular carcinoma patients after hepatectomy. Translational Oncology, 2022. 26.
9. Lee, I. C., et al., Evolutionary Learning-Derived Clinical-Radiomic Models for Predicting Early Recurrence of Hepatocellular Carcinoma after Resection. Liver Cancer, 2021. 10 (6): p. 572-582.

What is claimed is:

1. A method for predicting hepatocellular carcinoma (HCC) recurrence risk of a patient after a surgery, comprising:
obtaining one or more pre-operative computed tomography (CT) images from the patient's liver, wherein the CT images include at least hepatic arterial phase and porto-venous phase images including features associated with tumor lesions;
feeding the one or more pre-operative CT images into a multiphasic deep-learning model and training the multiphasic deep-learning model with the one or more pre-operative CT images;
generating a joint image-based representation of the pre-operative CT images by concatenating corresponding extracted image features; and
calculating a predicted risk score of the HCC based on the extracted image features for planning post-operative treatment and monitoring within a predetermined time period after the surgery.

2. The method of claim 1, further comprising step of segmenting the liver region in the one or more pre-operative CT images using a liver segmentation model before feeding the one or more pre-operative CT images into the multiphasic deep-learning model.

3. The method of claim 1, wherein the multiphasic deep-learning model is configured to extract image features from the one or more pre-operative CT images.

4. The method of claim 1, wherein the multiphasic deep-learning model comprises:
a first residual network architecture applied to the hepatic arterial phase images;
a second residual network architecture applied to the porto-venous phase images; and
a multiphasic residual-network random survival forest (RSF) model, the RSF model combines the extracted image features to generate the predicted risk score.

5. The method of claim 4, wherein each of the two residual network architectures comprises at least one stem block, at least one convolution block, and at least one identification block.

6. The method of claim 4, wherein the RSF model uses a log-rank splitting rule and a bootstrap method for analyzing right-censored survival data.

7. The method of claim 1, further comprising inputting pre-operative clinical parameters of the patient into the multiphasic deep-learning model.

8. The method of claim 7, wherein the RSF model combines the extracted image features with the pre-operative clinical parameters to generate the predicted risk score.

9. The method of claim 8, wherein the pre-operative clinical parameters are selected from age, sex, hepatitis B surface antigen status, hepatitis C virus antibody status, history of fatty liver, alpha fetoprotein level, and model for End-Stage Liver Disease (MELD) score.

10. The method of claim 9, wherein the pre-operative clinical parameters further comprise smoking status, comorbidities, use of antiviral therapy for hepatitis B, baseline blood test results including platelet count, prothrombin time, albumin, alpha fetoprotein levels, or a combination thereof.

11. The method of claim 1, wherein the multiphasic deep-learning model is optimized using the Adam optimizer with batch normalization.

12. The method of claim 1, wherein the predetermined time period ranges from 1 to 5 years.

13. A system for predicting a risk of hepatocellular carcinoma recurrence, the system comprising:
a processor;
a memory storing instructions, wherein the memory storing instructions, when executed by the processor, cause the system to perform the following steps:
obtaining one or more pre-operative computed tomography (CT) images from the patient's liver, wherein the CT images include at least hepatic arterial phase and porto-venous phase images that feature characteristics associated with tumor lesions;
feeding the one or more pre-operative CT images into a multiphasic deep-learning model and training the multiphasic deep-learning model with the one or more pre-operative CT images;
generating a joint image-based representation of the pre-operative CT images by concatenating corresponding image features; and
calculating a predicted risk score of the HCC based on a visual information of the joint image-based representation for planning post-operative treatment and monitoring within a predetermined time period after the surgery; and
a user interface to display an output of the predicted risk score.

14. The system of claim 13, wherein the memory storing instructions further cause the system to segment the liver region in the one or more pre-operative CT images using a liver segmentation model before feeding the one or more pre-operative CT images into the multiphasic deep-learning model.

15. The system of claim 13, wherein the multiphasic deep-learning model comprises:
an image model based on two residual network architectures, wherein the two residual network architectures are each applied separately to the hepatic arterial phase and porto-venous phase images;
a multiphasic residual-network random survival forest (RSF) model that combines the extracted image features to generate the predicted risk score.

16. The system of claim 15, wherein each of the two residual network architectures comprises at least one stem block, at least one convolution block, and at least one identification block.

17. The system of claim 15, wherein the RSF model uses a log-rank splitting rule and a bootstrap method for analysing right-censored survival data.

18. The system of claim 13, wherein the memory storing instructions further cause the system to inputting pre-operative clinical parameters of the patient into the multiphasic deep-learning model.

19. The system of claim 18, wherein the pre-operative clinical parameters comprise age, sex, hepatitis B surface antigen status, hepatitis C virus antibody status, history of fatty liver, alpha fetoprotein level, MELD score, or a combination thereof.

20. The system of claim 19, wherein the pre-operative clinical parameters further comprise smoking status, comorbidities, use of antiviral therapy for hepatitis B, baseline blood test results including platelet count, prothrombin time, albumin, alpha fetoprotein levels, or a combination thereof.
